(19) United States
(12) Patent Application Publication
(10) Pub. No.: US 2019/0307425 A1
(43) Pub. Date: Oct. 10, 2019

(54) ULTRASOUND IMAGING TRACKING CONTROLLED PRESENTATION

(71) Applicant: B-K Medical Aps, Herlev (DK)

(72) Inventors: Henrik JENSEN, Nordhavn (DK); Fredrik GRAN, Limhamn (SE); Svetoslav Ivanov MIKOLOV, Farum (DK)

(73) Assignee: B-K Medical Aps, Herlev (DK)

(21) Appl. No.: 15/949,315

(22) Filed: Apr. 10, 2018

(51) Int. Cl. A61B 8/00; G06T 17/00; G06T 3/60; G06T 7/70; A61B 8/12

(52) U.S. Cl. CPC A61B 8/466; A61B 8/463; A61B 8/54; G06T 17/00; G06T 2207/10136; G06T 7/70; A61B 8/4263; A61B 8/12; G06T 3/60

(57) ABSTRACT

An imaging system includes an ultrasound imaging probe, a display and a console. The console is electrically interfaced with the ultrasound imaging probe and the display. The console includes a rendering engine configured to visually present an ultrasound imaging image generated with data acquired by the ultrasound probe and a three-dimensional graphical representation of a portion of the probed superimposed over a predetermined region of the ultrasound image. The rendering engine is further configured to visually present the three-dimensional graphical representation in a spatial orientation of the probe with respect to a user.

### TECHNICAL FIELD

The following generally relates to ultrasound imaging and more particularly to visually presenting an ultrasound image with a three-dimensional (3-D) graphical representation of at least a portion of the ultrasound transducer used to generate the ultrasound image superimposed over a portion of the display in a spatial orientation corresponding to a current spatial orientation of the ultrasound transducer, which is determined by a probe tacking system, with respect to a user of the transducer.

### BACKGROUND

An ultrasound (US) imaging system has included an ultrasound probe with an array of transducer elements and interfaced with a console. The transducer elements transmit a pressure wave in response to being excited and sense echoes produced in response to the pressure wave interacting with structure and generates a signal indicative thereof. The console includes a processor that processes the signal to generate an image. In B-mode, the signal is processed to produce a sequence of focused, coherent echo samples along focused scanlines of a scanplane. The scanlines are scan converted into a format of a display monitor and visually presented as an image via the display monitor.

The probe housing has included a small protrusion near one side of the transducer array that protrudes out from the housing. The protrusion indicates a left/right orientation of the transducer array. By convention, the protrusion should point toward the patient's right side in transverse views and head in longitudinal views. The displayed image has been overlaid with an on-screen marking that corresponds to the protrusion. The side of the image corresponding with the protrusion end of the transducer is shown onscreen with an orientation marker. In this manner, the sonographer will be visually apprised of the image plane and orientation of the displayed image. An example is shown in FIG. 1.

FIG. 1 shows up/left 102, up/right 104, down/left 106 and down/right 108 orientations, each with an orientation marker 110. In practice, the displayed orientation is controlled by flipping the image up/down and left/right. Unfortunately, the displayed orientation is not very intuitive at least in that it provides little indication of the orientation of the transducer. For example, with a tightly curved transducer, the user has to look carefully to see if the image is flipped left/right. A user often still has to put their finger on the side the transducer to locate the protrusion to determine the orientation of the probe. With applications such as laparoscopy where the user cannot directly see the transducer, the user depends on the displayed image to understand the current orientation.

### SUMMARY

Aspects of the application address the above matters, and others.

In one aspect, an imaging system includes an ultrasound imaging probe, a display and a console. The console is electrically interfaced with the ultrasound imaging probe and the display. The console includes a rendering engine configured to visually present an ultrasound image generated with data acquired by the ultrasound imaging probe and a three-dimensional graphical representation of a portion of the probed superimposed over a predetermined region of the ultrasound image. The rendering engine is further configured to visually present the three-dimensional graphical representation in a spatial orientation of the probe with respect to a user.

In another aspect, a method includes acquiring scan data of a subject generated by an ultrasound imaging probe, processing the scan data to generate an ultrasound image, retrieving a three-dimensional representation including a 3-D graphical model of a probe, and visually presenting the ultrasound image with the three-dimensional graphical representation, including the 3-D graphical model of the probe and a scan plane, superimposed over the ultrasound image and in a spatial orientation of the ultrasound imaging probe with respect to a user of the ultrasound imaging probe.

In another aspect, a computer readable medium is encoded with computer executable instructions which when executed by a computer processor cause the computer processor to: acquire scan data of a subject generated by an ultrasound imaging probe, process the scan data to generate an ultrasound image, retrieve a three-dimensional representation including a 3-D graphical model of a probe, and visually present the ultrasound image with the three-dimensional graphical representation, including the 3-D graphical model of the probe and a scan plane, superimposed over the ultrasound image and in a spatial orientation of the ultrasound imaging probe with respect to a user of the ultrasound imaging probe.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 depicts a prior art display of images with a probe orientation marker;

FIG. 2 schematically illustrates an example ultrasound imaging system with a rendering engine the displays at least a 3-D representation of at least a portion of probe superimposed over an image and showing an orientation of the probe with respect to the sonographer;

FIG. 3 graphically illustrates an example of the 3-D representation and an image plane superimposed over a display showing an ultrasound image;

FIG. 4 graphically illustrates another example of the 3-D representation;

FIG. 5 graphically illustrates yet another example of the 3-D representation;

FIG. 6 graphically illustrates still another example of the 3-D representation;

FIG. 7 graphically illustrates another example of the 3-D representation with an anatomical model of the anatomy being scanned;

FIG. 8 graphically illustrates another example of the 3-D representation;

FIG. 9 graphically illustrates yet another example of the 3-D representation;

FIG. 10 graphically illustrates still another example of the 3-D representation;

FIG. 11 graphically illustrates another example of the 3-D representation;

FIG. 12 illustrates an example of the ultrasound imaging system of FIG. 2;

FIG. 13 illustrates a method in accordance with an embodiment(s) disclosed herein;

FIG. 14 illustrates another method in accordance with an embodiment(s) disclosed herein; and

FIG. 15 illustrates yet another method in accordance with an embodiment(s) disclosed herein.

### DETAILED DESCRIPTION

The following describes an approach that uses probe spatial tracking data to display a 3-D representation of at least part of an ultrasound probe in an orientation with respect to the sonographer, superimposed over part of a display with an ultrasound image of the scanned anatomy. The 3-D representation visually indicates the orientation of the ultrasound probe with respect to the sonographer.

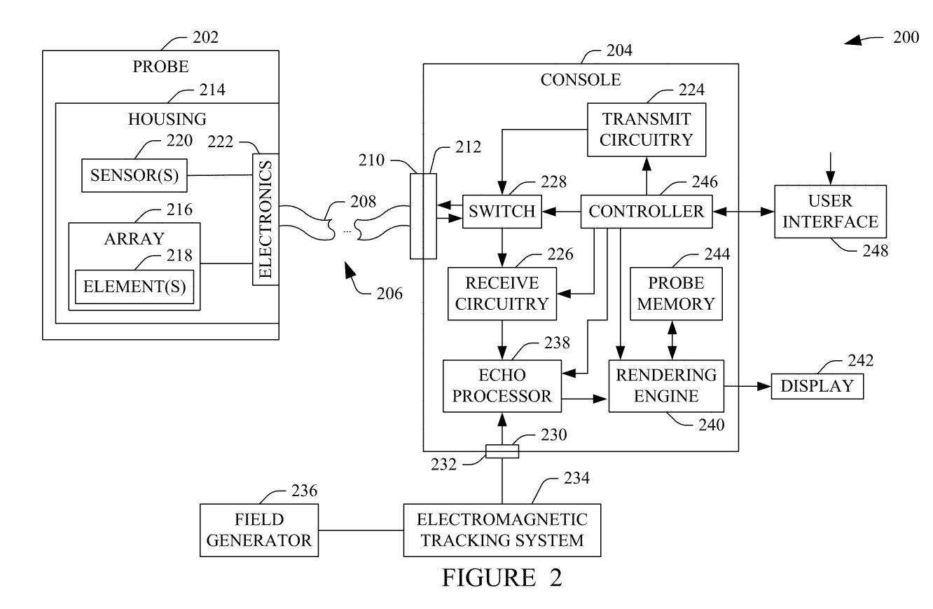

Initially referring to FIG. 2, an example imaging system 200, such as an ultrasound (US) imaging system, is schematically illustrated.

The imaging system 200 includes a probe 202 and a console 204, which are configured to interface over a communications path 206. In the illustrated example, the communications path 206 includes a hard-wired path 208 such as a cable or the like. In this instance, the cable 208 includes a connector 210 and the console 204 includes a complementary connector 212. In general, the console 204 may include multiple connectors, each configured to engage a complementary connector of a different probe. In another instance, the communications path 206 includes a wireless communications path, and the probe 202 and the console 204 include wireless interfaces.

In the illustrated example, the connectors 210 and 212 are electro-mechanical connectors. In one instance, the electro-mechanical connectors 210 and 212 are configured as plug and socket connectors, where the electro-mechanical connector 210 has a “male” configuration and is a plug with electrically conductive pins or prongs, and the complementary electro-mechanical connector 212 has a “female” configuration and is a mating receptacle with electrically conductive sockets configured to receive the pins or prongs. Mechanically engaging the electro-mechanical connectors 210 and 212 places the pins/prongs and sockets in electrical communication.

The probe 214 includes a housing 214, a transducer array 216 of transducer elements 218, a sensor(s) 220, and electronics 222. The housing 214 houses or encloses the transducer array 216, which is mechanically supported by and/or within the housing 214. The transducer array 216 includes one or more rows of the transducer elements 218, which are configured to transmit ultrasound signals and receive echo signals. The sensor(s) 220 includes one or more optical and/or electro-magnetic sensors and are used as discussed below for probe tracking purposes. The electronics 222 routes signals to and from the sensor(s) 220 and array 116 and the communications path 206. In one instance, the probe 202 transmits information that identifies the type (e.g., model) of the probe 202.

In a variation, the housing 214 includes a probe orientation marker. In one instance, the probe orientation marker is disposed at a predetermined location on the housing and visually and/or haptically indicates information such as the image plane and/or an orientation of the probe 202. An example of a suitable marker is described in patent application Ser. No. 15/513,216, publication number US 2017/303,892 A1, filed Mar. 22, 2017, and entitled “Transducer Orientation Market,” which is incorporated herein in its entirety by reference.

The console 204 includes transmit circuitry 224 configured to generate a set of radio frequency (RF) pulses that are conveyed to the transducer array 216 and selectively excite a set of the transducer elements 218, causing the set of elements to transmit ultrasound signals or pressure waves. The console 204 further includes receive circuitry 226 that receives electrical signals generated by the transducer elements 218 in response to the transducer elements 218 receiving echoes (RF signals) generated in response to the transmitted ultrasound signals or pressure waves interacting with structure (e.g., organ cells, blood cells, etc.).

The console 204 further includes a switch 228 that switches between the transmit circuitry 224 and the receive circuitry 226, depending on whether the transducer array 216 is in transmit mode or receive mode. In transmit mode, the switch 228 electrically connects the transmit circuitry 224 to the transducer array 216 and hence the transducer elements 218. In receive mode, the switch 228 electrically connects the receive circuitry 226 to the transducer array 216 and hence the transducer elements 218. In a variation, separate switches are used for transmit and receive operations.

The console 204 further includes an interface 230 to a complementary interface 232 of an electromagnetic tracking system 234, which includes a field generator 236. The interfaces 230 and 232 can be electro-mechanical connectors, e.g., similar to the connectors 210 and 212 described herein. The electromagnetic tracking system 234 and the sensor(s) 220 together track the spatial position of the probe 202. The electromagnetic tracking system 234 conveys a signal indicative of this position to the console 204 via the interfaces 230 and 232. A suitable example of the electromagnetic tracking system 234 is the Aurora tracking system, a product of NDI, which is headquartered in Ontario, Canada. In a variation, an optical tracking system in employed.

The console 204 further includes an echo processor 238 that processes the electrical signals from the receive circuitry 226. In one instance, such processing includes applying time delays, weighting on the channels, summing, and/or otherwise beamforming received electrical signals. In B-mode, the echo processor 238 produces a sequence of focused, coherent echo samples along focused scanlines of a scanplane. The echo processor 238 further synchronizes the tracking signal from the tracking system 234 with the beamformed image such that the spatial orientation of the probe 202 for each image is linked to the image.

The console 204 further includes a rendering engine 240 and a display monitor 242. The rendering engine 240 is configured to displays images via the display 212. In one instance, this includes displaying an ultrasound image with a graphical representation of the probe 202 and, optionally, a scan plan superimposed over the ultrasound image in a region outside of the scanned anatomy. In one instance, the probe 202 in the graphical representation visually resembles the probe 202. The type (e.g., model) of the probe 202 is ascertained by the identification signal from the probe 202 and/or user input identifying the type of the probe 202. In a variation, a same graphical representation is used for all probes. In this instance, the probe type is not provided and/or indicated.

A probe memory 244 stores models of each type of probe 202 that can be used with the console 204. The rendering engine 240 retrieves a model based on the identification of the type of probe 202, a default, a user preference, etc. Each model is a three-dimensional (3-D) model and is displayed as a 3-D graphical representation of the probe 202 oriented on the display 242 based on the tracking signal such that the displayed 3-D graphical representation of the probe 202 reflects a spatial orientation of the probe 202 with respect to the sonographer. The 3-D graphical representation moves (e.g., rotates, translates, etc.) with the probe 202 so that it represents a current spatial orientation of the probe 202.

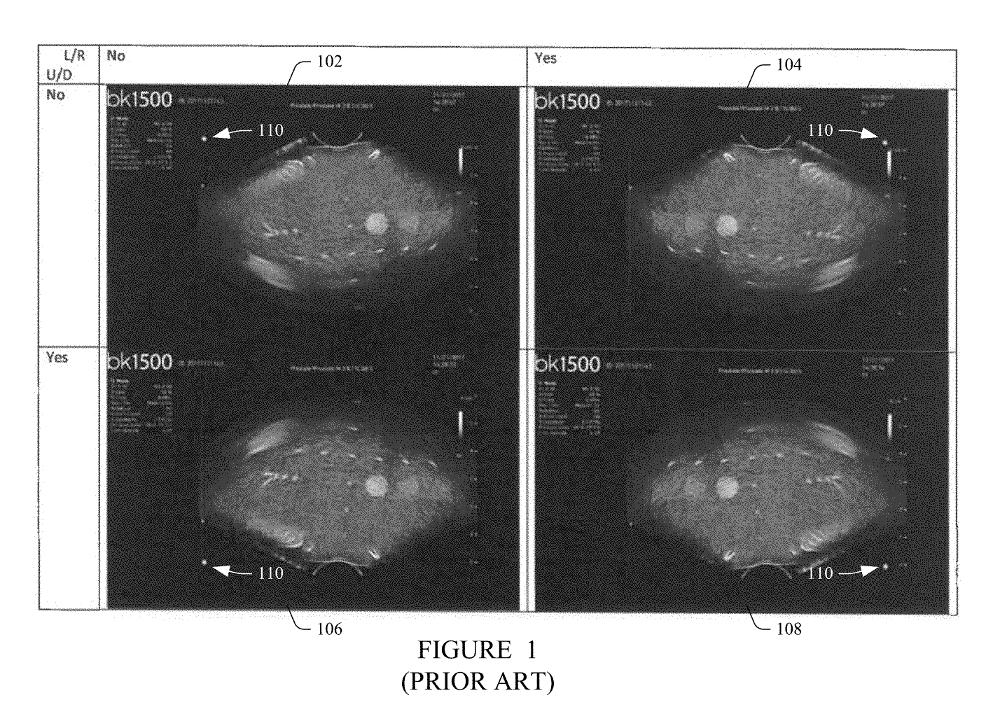

In one instance, this provides for a more intuitive display, e.g., relative to the display shown in FIG. 1. As such, in one instance the user need not have to look at the probe and/or put their finger on the orientation protrusion and/or logo to determine the orientation of the probe 202 with respect to the sonographer. This is well-suited for applications where the probe 202 is not readily visible such as in the case in laparoscopy where the user cannot directly see the transducer, and the sonographer depends on the displayed 3-D graphical representation to understand the current orientation of the probe 202.

The console 204 further includes a controller 246 (which includes a processor, etc.) The controller 246 controls one or more of the components 212-244. Such control, in one instance, is based on a selected and/or activated visualization mode. For example, when a first visualization mode is active, the rendering engine 240 is provided with the type of the probe 202 and uses this information along with the probe tracking signal, the retrieve a suitable 3-D probe model and display the ultrasound image with the 3-D graphical representation of the probe 202 as described herein. In another visualization mode, the 3-D graphical representation is not displayed.

The console 204 also interfaces a user interface (UI) 248. The UI 248 includes one or more input devices (e.g., buttons, knobs, trackball, etc.) and/or one or more output devices (e.g., visual, audio, etc. indicators). The UI 248 can be used to select an imaging mode, a visualization mode (e.g., the first visualization mode), etc. As discussed herein, in one instance the user identifies the type of the probe 202 being used. In this instance, the user employs the UI 248 to make the selection. For example, the user can employ of pointing device (e.g., a mouse) of the US 248 to select the probe types from a menu or list of available probe types, manually enter via a keyboard the probe type, etc.

It is to be appreciated that the echo processor 238 and/or the rendering engine 240 are implemented by a processor such a central processing unit, a microprocessor, etc. In this instance, the console 2047 further includes computer readable medium (which excludes transitory medium and includes physical memory) encoded with computer executable instructions. The instructions, when executed by the processor, cause the processor to perform one or more of the functions described herein.

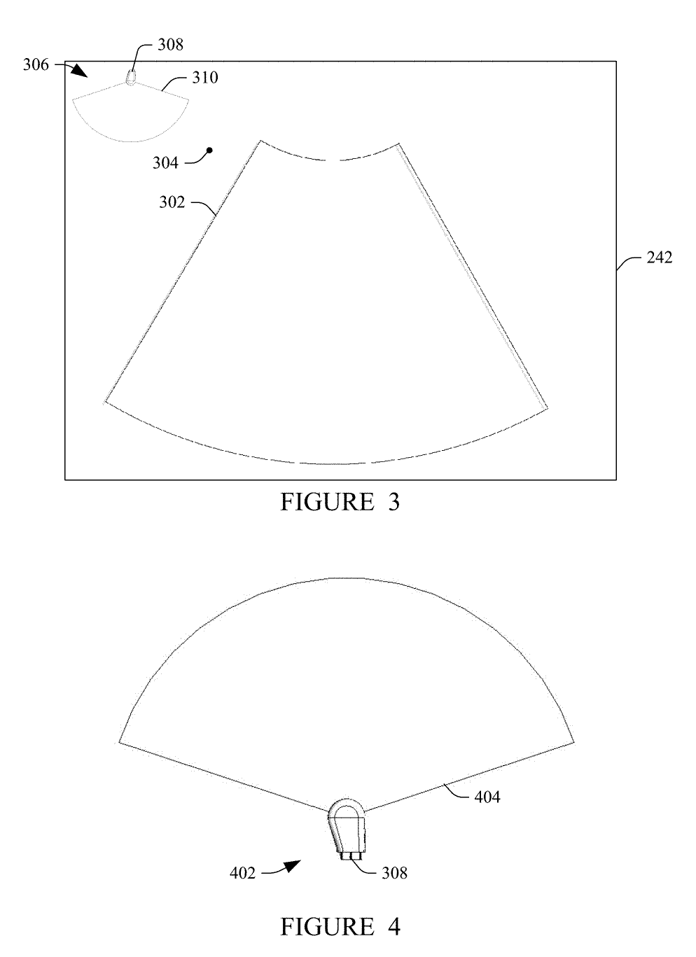

FIG. 3 schematically illustrate an example of the displayed information. An image 302 is displayed in the display 242 in a down/left orientation, which is indicated by an orientation maker 304. A 3-D graphical representation 306 is shown in a top right corner of the display 242. This location is for explanatory purposes and is not limited; the 3-D graphical representation 306 is positional anywhere on the display. The illustrated 3-D graphical representation 306 includes a portion 308 of the probe 202 and an image plane 310.

In this example, the 3-D graphical representation 306 shows the probe 202 is currently facing down with respect to the sonographer. The 3-D graphical representation 306 moves with the probe 202 with continuous tilt in the plane of the display 242 and three hundred and sixty degrees (360°) rotation into and out of the plane with the probe 202 so that it always reflects a current 3-D orientation of the probe 202 with respect to the sonographer.

FIG. 4 schematically illustrates another example of the displayed information. In this example, a 3-D graphical representation 402 includes the portion 308 of the probe 202 and an image plane 404. In this example, the 3-D graphical representation 402 shows the probe 202 is currently facing up with respect to the sonographer.

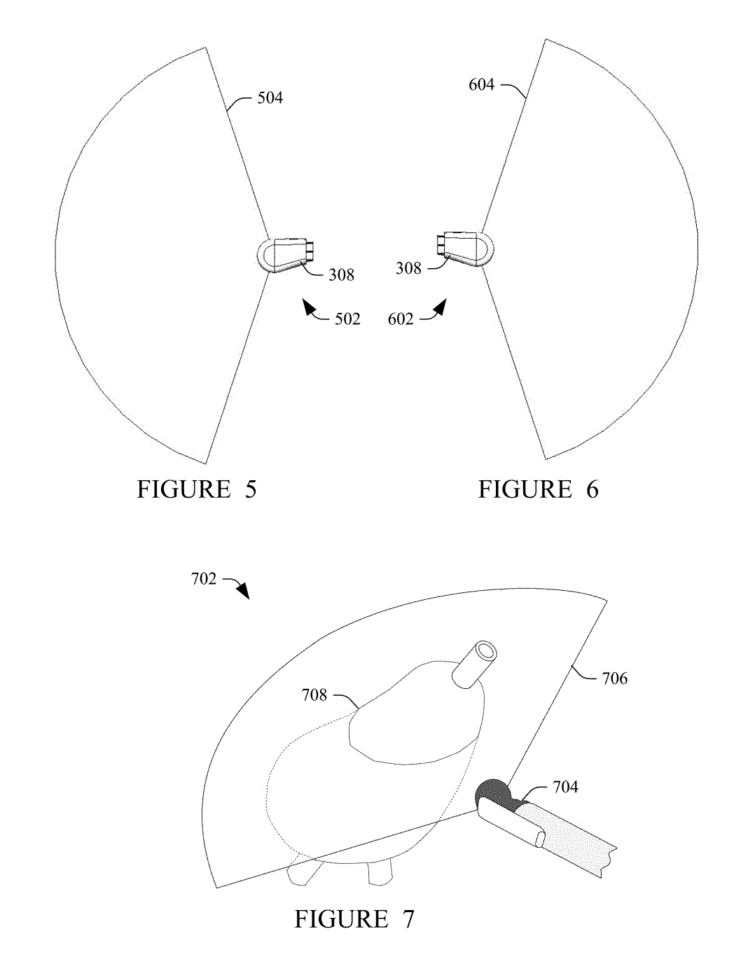

FIG. 5 schematically illustrates another example of the displayed information. In this example, a 3-D graphical representation 502 includes the portion 308 of the probe 202 and an image plane 504. In this example, the 3-D graphical representation 504 shows the probe 202 is currently facing left with respect to the sonographer.

FIG. 6 schematically illustrates another example of the displayed information. In this example, a 3-D graphical representation 602 includes the portion 308 of the probe 202 and an image plane 604. In this example, the 3-D graphical representation 604 shows the probe 202 is currently facing right with respect to the sonographer.

FIG. 7 schematically illustrates a variation where the tracking information also includes information about the anatomy being scanned. This example is for an end-fire probe. In this example, a 3-D graphical representation 702 includes a portion 704 of the probe 202, an image plane 706, and 3-D graphical representation 708 (e.g., a model, an atlas, etc.) of the anatomy being scanned.

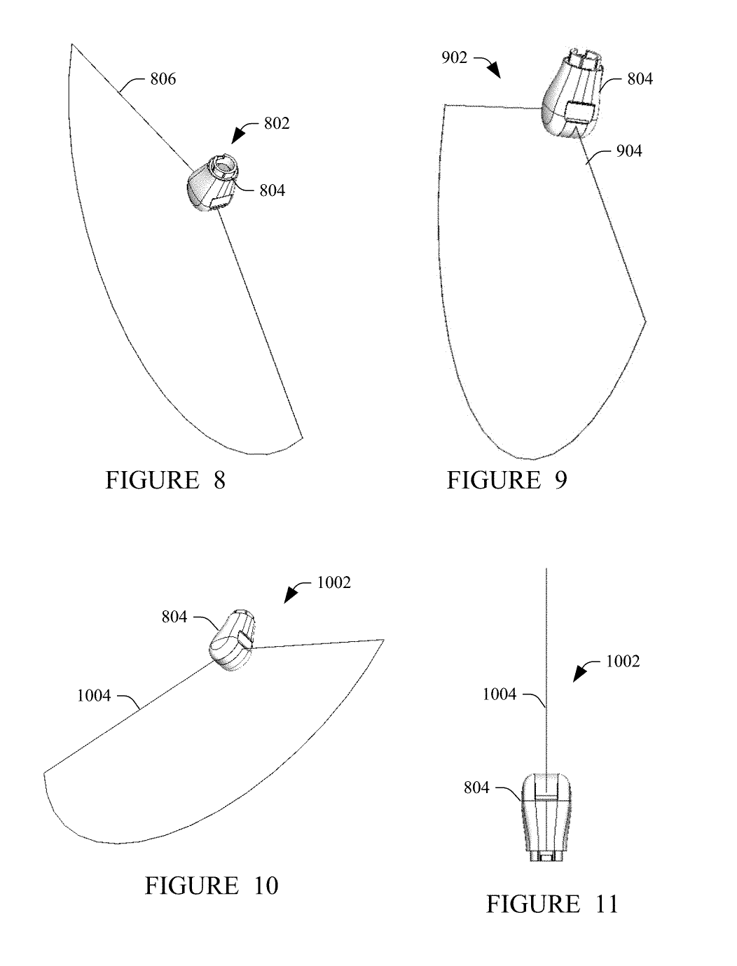

FIGS. 8-11 schematically illustrate other example of the displayed information. In FIG. 8, a 3-D graphical representation 802 includes a portion 804 of the probe 202 and an image plane 806. In FIG. 9, a 3-D graphical representation 902 includes the portion 804 of the probe 202 and an image plane 904. In FIG. 10, a 3-D graphical representation 1002 includes the portion 804 of the probe 202 and an image plane 1004. In FIG. 11, a 3-D graphical representation 1102 includes the portion 804 of the probe 202 and an image plane 1104.

For probes with more than one transducer array (e.g., a bi-plane probe), in one instance, the 3-D representation simultaneously shows both scan planes. In another instance, the 3-D representation shows only a one scan plane at a time. In this instance, the user can toggle between the scan planes. In another instance, the user selects to show the scan planes simultaneously and/or individually.

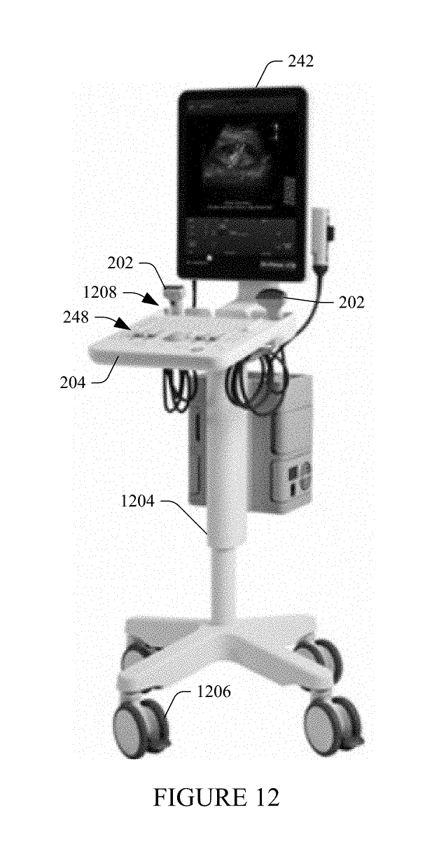

FIG. 12 illustrates a non-limiting example of the ultrasound imaging system 200. In this example, the console 204 is affixed to a mobile cart 1204, which include movers 1206 such as wheels, casters, etc., the user interface 248 is part of console 104, and the display 242 is affixed to the mobile cart 1204. In another configuration, the ultrasound imaging system 200 does not include movers, but instead is configured to rest on a table, desk, etc. The console 204 includes at least one holder 1208 configured to support at least one transducer probe, such as the probe 202.

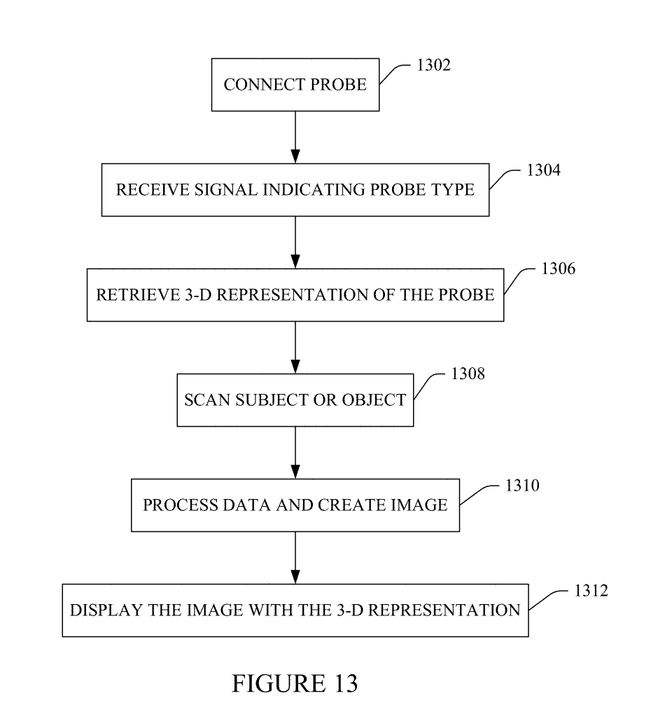

FIG. 13 illustrates a method in accordance with an embodiment(s) disclosed herein.

At 1302, the probe 202 is connected to the console 204.

At 1304, the probe 202 transmits a signal indicating its type.

At 1306, the rendering engine 240 retrieves a 3-D representation of the probe 202 based on the signal.

At 1308, the probe 202 is used to scan a subject or object.

At 1310, the echo processor generates an ultrasound image from the acquired data.

At 1312, the rendering engine 240 displays the ultrasound image with the 3-D representation of the probe 202 showing a spatial orientation of the probe 202 with respect to the sonographer, as discussed herein and/or otherwise.

Acts 1308, 1310 and 1312 can be repeated one or more times.

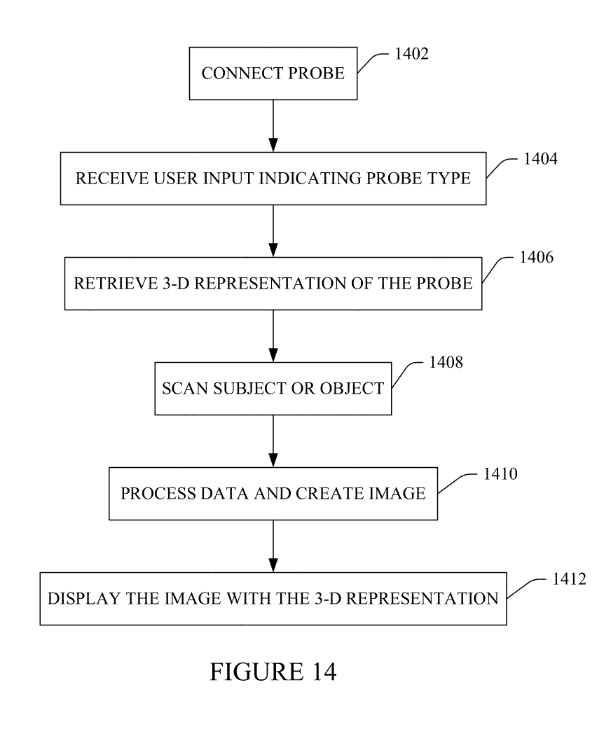

FIG. 14 illustrates another method in accordance with an embodiment(s) disclosed herein.

At 1402, the probe 202 is connected to the console 204.

At 1404, a user indicates the type of probe 204 with the user interface 248.

At 1406, the rendering engine 240 retrieves a 3-D representation of the probe 202 based on the signal.

At 1408, the probe 202 is used to scan a subject or object.

At 1410, the echo processor generates an ultrasound image from the acquired data.

At 1412, the rendering engine 240 displays the ultrasound image with the 3-D representation of the probe 202 showing a spatial orientation of the probe 202 with respect to the sonographer, as discussed herein and/or otherwise.

Acts 1408, 1410 and 1412 can be repeated one or more times.

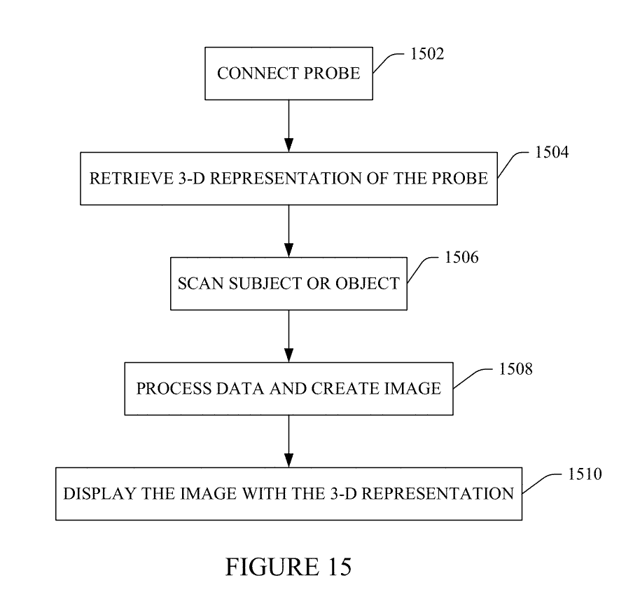

FIG. 15 illustrates yet another method in accordance with an embodiment(s) disclosed herein.

At 1502, the probe 202 is connected to the console 204.

At 1504, the rendering engine 240 retrieves a 3-D representation of a probe.

At 1506, the probe 202 is used to scan a subject or object.

At 1508, the echo processor generates an ultrasound image from the acquired data.

At 1510, the rendering engine 240 displays the ultrasound image with the 3-D representation of the probe showing a spatial orientation of the probe 202 with respect to the sonographer, as discussed herein and/or otherwise.

Acts 1506, 1508 and 1510 can be repeated one or more times.

At least a portion of the method(s) discussed herein may be implemented by way of computer readable instructions, encoded or embedded on computer readable storage medium (which excludes transitory medium), which, when executed by a computer processor(s), causes the processor(s) to carry out the described acts. Additionally, or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

What is claimed is:

1. An imaging system, comprising:
an ultrasound imaging probe;
a display; and
a console electrically interfaced with the ultrasound imaging probe and the display and including a rendering engine configured to visually present an ultrasound image generated with data acquired by the ultrasound imaging probe and a three-dimensional graphical representation of a portion of the probed superimposed over a predetermined region of the ultrasound image, wherein the rendering engine is further configured to visually present the three-dimensional graphical representation in a spatial orientation of the probe with respect to a user.

2. The imaging system of claim 1, wherein the console is configured to receive a signal indicative of the spatial orientation of the probe from a remote probe tracking system in electrical communication with the console.

3. The imaging system of claim 2, wherein the ultrasound probe includes a tracking sensor configured to communicate the probe tracking information to the remote probe tracking system.

4. The imaging system of claim 1, wherein the three-dimensional graphical representation further includes an image plane.

5. The imaging system of claim 4, wherein the three-dimensional graphical representation further includes an anatomical model or atlas of scanned anatomy.

6. The imaging system of claim 1, wherein the rendering engine is configured to update the three-dimensional graphical representation so that the displayed orientation tracks a current orientation of the probe as the ultrasound imaging probe is moved.

7. The imaging system of claim 1, wherein the rendering engine is configured to at least one of tilt the three-dimensional graphical representation in a plane of the display and rotate the three-dimensional graphical representation in and out of the plane in coordination with moving the ultrasound imaging probe so that the displayed orientation tracks a current orientation of the ultrasound imaging probe as the ultrasound imaging probe is moved.

8. The imaging system of claim 1, wherein the ultrasound imaging probe is configured to transmit information identifying a type of the ultrasound imaging probe to the console, and further comprising:
a probe memory configured to store models of different types of ultrasound imaging probes; and
a controller configured to transmit the type of the ultrasound imaging probe to the rendering engine, wherein the rendering engine selects a model from the probe memory in accordance with the type of the ultrasound imaging probe, and the three-dimensional graphical representation includes the model.

9. The imaging system of claim 1, further comprising:
a user interface configured to receive an input identifying a type of the ultrasound imaging probe;
a probe memory configured to store models of different types of probes; and
a controller configured to transmit the type of the ultrasound imaging probe to the rendering engine, wherein the rendering engine selects a model from the probe memory in accordance with the type of the ultrasound imaging probe, and the three-dimensional graphical representation includes the model.

10. The imaging system of claim 1, further comprising:
a probe memory configured to store a model of a probe, wherein the rendering engine retrieves the model of the ultrasound imaging probe from the probe memory, and the three-dimensional graphical representation includes the model.

11. A method, further comprising:
acquiring scan data of a subject generated by an ultrasound imaging probe;
processing the scan data to generate an ultrasound image;
retrieving a three-dimensional representation including a 3-D graphical model of a probe; and
visually presenting the ultrasound image with the three-dimensional graphical representation, including the 3-D graphical model of the probe and a scan plane, superimposed over the ultrasound image and in a spatial orientation of the ultrasound imaging probe with respect to a user of the probe.

12. The method of claim 11, further comprising:
receiving an identification of the ultrasound imaging probe from the ultrasound imaging probe, wherein the 3-D graphical model corresponds to the identification of the ultrasound imaging probe.

13. The method of claim 11, further comprising:
receiving an identification of the ultrasound imaging probe from a user interface interfaced with an ultrasound console, wherein the 3-D graphical model corresponds to the identification of the ultrasound imaging probe.

14. The method of claim 11, wherein a same 3-D graphical model is retrieved regardless of the type of the ultrasound imaging probe.

15. The method of claim 11, further comprising:
receiving, from a tracking device, a tracking signal indicative of a spatial orientation of the ultrasound imaging probe; and
visually presenting the 3-D graphical model in a spatial orientation corresponding the tracking signal.

16. The method of claim 15, further comprising:
at least one of rotating and translating the 3-D graphical model based on the tracking signal so that the visually presented orientation tracks a current orientation of the ultrasound imaging probe as the ultrasound imaging probe is moved.

17. A computer readable medium encoded with computer executable instructions which when executed by a computer processor cause the computer processor to:
acquire scan data of a subject generated by an ultrasound imaging probe;
process the scan data to generate an ultrasound image;
retrieve a three-dimensional representation including a 3-D graphical model of a probe; and
visually present the ultrasound image with the three-dimensional graphical representation, including the 3-D graphical model of the probe and a scan plane, superimposed over the ultrasound image and in a spatial orientation of the ultrasound imaging probe with respect to a user of the ultrasound imaging probe.

18. The computer readable medium of claim 17, further comprising:
receive, from a tracking device, a tracking signal indicative of a spatial orientation of the ultrasound imaging probe; and
visually present the 3-D graphical model in a spatial orientation corresponding the tracking signal.

19. The computer readable medium of claim 18, further comprising:
at least one of rotate and translate the 3-D graphical model based on the tracking signal so that the 3-D graphical model is visually presented in an orientation that tracks a current orientation of the ultrasound imaging probe as the ultrasound imaging probe is moved.

20. The computer readable medium of claim 18, further comprising:
receive, from the tracking device, a second tracking signal indicative of a spatial orientation of scanned anatomy; and
visually present the scanned anatomy with the 3-D graphical model and in a spatial orientation corresponding the tracking signal.
